(19) United States
(12) Patent Application Publication
(10) Pub. No.: US 2022/0305476 A1
(43) Pub. Date: Sep. 29, 2022

(54) IMPROVED METHOD FOR THE CATALYZED HYDROISOMERISATION OF HYDROCARBONS

(71) Applicant: Clariant International Ltd, Muttenz (CH)

(72) Inventors: Rainer Albert RAKOCZY, Buchloe (DE); Johannes HARDER, München (DE)

(21) Appl. No.: 17/641,491

(22) PCT Filed: Sep. 4, 2020
(86) PCT No.: PCT/EP2020/074823

§ 371 Date: Mar. 9, 2022

(30) Foreign Application Priority Data
Sep. 13, 2019 (DE) 10 2019 124 731.4

(51) Int. Cl. B01J 35/00; B01J 23/42; B01J 29/04; C07C 5/27; B01D 3/00

(52) U.S. Cl. CPC B01J 35/0006; B01J 23/42; B01J 29/043; C07C 5/2775; B01D 3/009; C07C 2521/04; C07C 2523/42; C07C 2529/068

(57) ABSTRACT

The invention relates to an arrangement of several layers of catalysts arranged in series in a reactor for the hydroisomerisation of hydrocarbons, to a method for the hydroisomerisation of hydrocarbons and to the use of the arrangement for the hydroimerisation of hydrocarbons.

The present invention relates to an arrangement of multiple successive layers of catalysts in a reactor for the hydroisomerisation of hydrocarbons, and also to a process for hydroisomerisation of hydrocarbons and to the use of this arrangement for the hydroisomerisation of hydrocarbons.

Catalytic hydroisomerisation is an important process step for utilization of carbonaceous resources to give products such as fuels or commodity chemicals in the chemical and petrochemical industries. Sources for carbon or the corresponding hydrocarbons are hard coal tar, distillates and condensates from the coking of coal, natural gas, associated petroleum gas, crude oil, biomass, waste and especially plastic waste.

Many of these sources still contain compounds having heteroatoms such as oxygen, nitrogen and sulfur. Especially in the case of sulfur-containing sources, for example crude oil or hard coal tar, the sulfur compounds and other heteroatom compounds are desulfurized by hydroconversion, for example over NiMo, CoMo or NiW catalysts. In combination with additional, usually bifunctional catalysts, bond cleavages (cracking) or rearrangement reactions (isomerization) of the hydrocarbon compounds are possible under hydrogenating conditions. The purpose of this further conversion is, for example, the adjustment of a boiling range (hydrocracking) or of viscosity (deparaffinization, also called dewaxing).

In the field of mineral oil processing, there are processes for hydroisomerisation of already desulfurized hydrocarbon streams using bifunctional catalysts using precious metals, for example
isomerization of n-butane to isobutane over chlorinated aluminium chloride,
isomerization of pentane- and hexane-rich light gasoline cuts over zeolites,
isomerization of higher alkanes to isoalkanes over zeolites (C7+ isomerisation),
isomerization of cyclohexane-rich light gasoline cuts to methylcyclopentane.

Bifunctional catalysts in the context of the present invention are understood to mean supported catalysts, the supports of which, in the form of extrudates, spheres, tablets or other aggregates, as well as the catalytic activity of a metal component, have additional catalytic activity that can be generated by mixing in further components or using an already active uniform support material. In most cases, these are solid-state compounds having acidic or basic properties, such as zeolites, hydrotalcites, active mixed oxides in the broadest sense, but also ionic liquids or complexes.

Especially the isomerisation of light gasoline fractions is an important industrial scale process which is an essential step, inter alia, for the increasing of what is called the knock resistance of gasoline, in order to prevent uncontrolled self-ignition of the fuel in the engine.

With growing demand for sulfur-free diesel (ULSD) with a maximum sulfur content of 10 ppm by mass, most refineries require separate hydrogen production by conventional steam reforming. The original source of hydrogen, the semi-regenerative catalytic reformer (CRU), or continuous catalytic reformers (CCR) with heavy light gasoline as feed stream are no longer adequate. The availability of an additional and far more efficient hydrogen source in a refinery permits a distinctly more economically viable mode of operation of the catalytic reformers. Depending on the quality of the crude oil, it is possible to obtain untreated native (straight-run) light gasoline streams in an order of magnitude of 15% to 25% by mass directly by atmospheric distillation out of the crude oil. Depending on the degree of complexity and the level of utilization of heavier fractions from crude oil distillation, it is possible to increase the proportion of boiling fractions in the gasoline range in a refinery up to 50% by mass.

The baseload for achievement of the necessary knock resistance for straight-run light gasoline streams is borne primarily by the operation of the catalytic reformers. The knock resistance of the lighter components (C₅ and C₆) is increased via the isomerisation. The isomerisation is generally the very last tool for additional optimization of the yield of gasoline itself in very complex refineries. By virtue of the availability of additional hydrogen from steam reforming, it is nowadays possible to match the interplay of catalytic reformer and isomerisation more and more with a view to knock resistance, vapour pressure and economic viability.

As a result of greater limitation in the benzene content in the gasoline, isomerisation is nowadays an ever more important method for additionally and in some cases even primarily exploiting the hydrogenating properties of the precious metal component of the catalyst for the saturation of benzene.

In addition, more and more plant operators are tending to blend lighter fractions from the catalytic reformer into the isomerisation feed. This results in the presence of olefins and diolefins in the isomerisation feed oil. These very active compounds have a very adverse effect on the process of isomerisation.

Hidalgo et al. (Eur. J. Chem., 12(1), 2014, p. 1-13) discloses various processes for hydroisomerisation in which the reaction fluid is guided into a reactor containing a hydroisomerisation catalyst.

Regardless of the choice of the isomerisation processes, there are additional variants for optimization of the octane number of the products that generally involve separating branched or cyclic hydrocarbons from the reactant or product stream and recycling the unbranched hydrocarbons in order to enrich these in the reactant stream that is guided into the reactor for the hydroisomerisation step. This is accomplished by distillation or adsorption (E. A. Yasakova, A. V. Sitdikova, A. F. Achmetov, TENDENCY OF ISOMERIZATION PROCESS DEVELOPMENT IN RUSSIA AND FOREIGN COUNTRIES, Oil and Gas Business (2010)).

The further variant described in U.S. Pat. No. 5,948,948 A involves performing the hydroisomerisation by subjecting the process stream to a reactive distillation.

The bifunctionally catalysed hydroisomerisation is an equilibrium reaction wherein the direction of reaction toward the desired isoalkanes is preferred at lower temperatures. Since the by-products present in the reaction fluid are also converted in exothermic reactions under the reaction conditions and in the presence of the catalyst for the hydroisomerisation, this increases the reaction temperature, which reduces the selectivity for the desired isoalkanes. Moreover, as a result of the catalytic composition of the by-products for the desired hydroisomerisation, there are fewer free catalytic sites available, which likewise has an adverse effect on the selectivity for the desired isoalkanes.

Native light gasoline fractions may contain up to 5% by weight of aromatics such as benzene and toluene. These may be hydrogenated under the process conditions, which results in an additional increase in temperature within the reactor and additionally moves the equilibrium disadvantageously. The same is true in the presence of mercaptans.

The presence of organic nitrogen compounds, especially amines, has two effects on the catalytic activity of a hydroisomerisation catalyst. For instance, there is conversion of the amine to ammonia at the platinum function, which is a competing reaction to the desired initiation of the isomerisation. Moreover, these compounds are basic in nature, and there is interaction with the acidic sites and hence a massive decrease in the activity of the catalyst. The conversion of the amines to ammonia reduces the passivating effect because the basicity of ammonia is distinctly lower than that of amines

As well as the unfavourable influence on the equilibrium position by the extreme rise in temperature associated with loss of RON (research octane number), the high rise in temperature also constitutes a substantial impairment of plant safety.

There was therefore the need for a process for hydroisomerisation of hydrocarbons with which more efficient conversion is possible and which also permits a safe mode of operation.

This problem is solved by the arrangement of the invention and a process utilizing the arrangement of the invention.

One subject of this invention relates to an arrangement of at least two successive layers of catalysts in a reactor for the hydroisomerisation of hydrocarbons. Further subjects relate to a process for hydroisomerisation of hydrocarbons and to the use of the arrangement for the hydroisomerisation of hydrocarbons.

In the arrangement of the invention, the first catalyst layer arranged upstream is chosen such that there is primarily hydrogenation of the stream of matter therein. The catalyst chosen in the second layer arranged downstream is a catalyst that brings about hydroisomerisation of the product stream.

A layer arranged upstream in the context of the present invention is understood to mean that layer through which the reaction fluid is guided first, whereas the layer arranged downstream is understood to mean that layer through which the reaction fluid is guided subsequently.

In one embodiment, the reactor is an adiabatically operated reactor. In the context of the present invention, “adiabatically operated” means that the conditions within the reactor are adiabatic or virtually adiabatic.

By utilization of the arrangement of the invention, it is possible to selectively hydrogenate the by-products in the first catalyst layer in order that they cannot enter into, or at least enter into a significantly lower level of, unwanted side reactions, for example the dimerization of olefins, saturation of aromatics with corresponding heats of reaction or the inhibition of the precious metal catalysts by the reaction with basic amines, in the downstream catalyst layer under the conditions of the hydroisomerisation and in the presence of the precious metal catalysts.

A reactor in the context of the present invention may be a single reactor housing. In another embodiment, the reactor may consist of multiple reactor housings arranged in succession.

The catalyst layers may either be present in the same reactor housing or they are arranged separately from one another in reactor housings arranged in succession.

There may additionally be layers of inert materials positioned above, between and/or beneath the catalyst layers. These may take the form of fixed reactor internals or of beds of inert material. These layers may serve to achieve better distribution of the components of the reaction fluid in the reactor, or to prevent catalyst material introduced into the reactor from falling out of it. In a preferred embodiment, the inert material is present beneath the second catalyst layer.

Suitable inert materials are preferably aluminium oxide, ceramics, fired silica or fireclay.

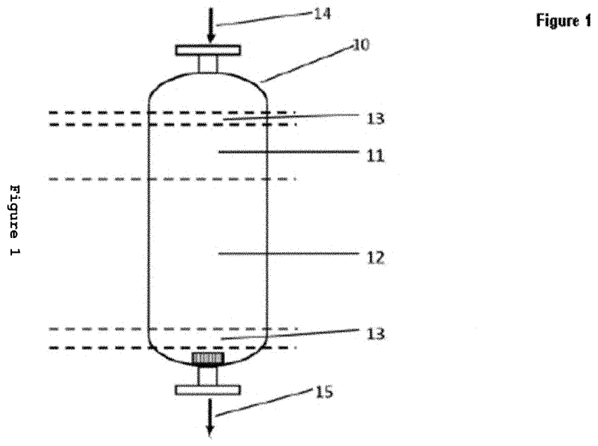

FIG. 1 shows a schematic diagram of an arrangement of the invention. In a reactor (10), there is a catalyst layer (11) arranged upstream, followed downstream by a further catalyst layer (12). In FIG. 1, there are also inert materials (13) above both the catalyst layer arranged upstream and the catalyst layer arranged downstream. In this diagram, the reaction fluid is introduced (14) into the reactor (11) from the top and discharged again (15) at the lower end.

Beyond the catalyst layer arranged downstream or the layer of inert material optionally arranged beyond it, there may also be one or more further catalyst layers.

For example, this further catalyst layer may comprise a catalyst for hydrodesulfurization in order to remove sulfur impurities present.

The first layer catalyst consists of a porous support to which a precious metal component has been applied. This is typically in metallic form. In a preferred embodiment, the precious metal component is selected from one of the elements Au, Pt, Rh, Pd, Ir, Ag, or mixtures thereof.

The precious metal component is typically applied by immersion of the porous support into a precious metal-containing solution, by the application of a precious metal-containing solution or suspension, or by what is called incipient wetness impregnation of a precious metal-containing solution.

The precious metal content of this catalyst may be within a range from 0.05% to 5.0% by weight, preferably from 0.1% to 4.0% by weight and more preferably from 0.1% to 3.0% by weight, based on the weight of the catalyst after ignition loss at 900° C.

The porous support of the catalyst in the first layer is typically a material selected from the list of aluminium oxide, silicon oxide, silicon-aluminium oxides, ceramic, metal foams and thermally stable polymers. The support has only slightly acidic or slightly basic properties. Such a support has very substantially no cracking and isomerisation activity. Thus, in one embodiment, the number of acidic sites determined by temperature-programmed desorption of ammonia (NH₄-TPD) is below 100 μmol/g, preferably below 50 μmol/g. For the determination of the acidity, 1-2 g of the sample in the form of a grain fraction of 200-400 μm is heated up to 550° C. under an He stream, then cooled down to 110° C., and an NH₃ stream in helium is passed over the sample at that temperature. Once the sample has been saturated with NH₃, the excess NH₃ is first purged out of the sample space. Subsequently, the sample is heated to 750° C. and the desorbing NH₃ is detected by mass spectrometer (mass number 16).

As described in Roessner et al. (N. Supamathanon, J. Wittayakun, S. Prayoonpokarach, W. Supronowicz and F. Roessner, Quim. Nova, vol. 35, no. 9, 1719-1723, 2012), a support with weakly basic properties can be characterized by its capacity to convert 2-methyl-3-butyn-2-ol to acetone or acetylene. In the context of the present invention, for this purpose, 20 mg of the sample is charged in a fixed bed reactor and heated under a nitrogen stream at 350° C. for 4 h. Subsequently, the sample is cooled down to 120° C. and, at that temperature, a gas stream consisting of 95% by volume of 2-methyl-3-butyn-2-ol and 5% by volume of toluene is passed through the reactor. Using the analysis of the gas stream downstream of the reactor by means of gas chromatography, it is possible to calculate the overall selectivity for acetone and acetylene. If this overall selectivity has a value of less than 30%, preferably less than 20%, the support is a weakly basic support for the purposes of the present invention.

In one embodiment, the support has a pore volume, determined by means of Hg porosimetry to DIN 66133, of at least 100 mm³/g, preferably at least 200 mm³/g and very preferably at least 300 mm³/g. In a further embodiment, the support has a pore volume, determined by means of Hg porosimetry to DIN 66133, of at most 800 mm³/g, preferably of at most 500 mm³/g. In a further embodiment, the support has a pore volume in the range from 100 to 800 mm³/g, preferably in the range from 200 to 500 mm³/g.

The support of this catalyst can be generated by extrusion, tableting, spherization, pelletization, injection moulding or 3D printing methods.

The catalyst for the second downstream layer is a bifunctional catalyst consisting of a porous acidic or basic support and a precious metal component. In a preferred embodiment, the precious metal component is selected from one of the elements Au, Pt, Rh, Pd, Ir, Ag, Re or mixtures thereof.

The precious metal component is typically applied by immersion of the porous support into a precious metal-containing solution, by the application of a precious metal-containing solution or suspension, or by what is called incipient wetness impregnation of a precious metal-containing solution.

The support for this catalyst consists of an acidic or basic active component and a binder. Preferred binders are aluminium oxide, for example pseudoboehmite, boehmite or corundum, silica, amorphous aluminosilicate, or aluminium oxides such as bentonite, or mixtures thereof. Preferred active components are zeolites, chlorinated aluminium oxide, tungstenated zirconium oxide or sulfonized zirconium oxide or mixtures thereof. Suitable zeolites are those having the following framework structure: ETR, VFI, AET, SFH, SFN, AFI, AFR, AFS, AFY, ATO, BEA, BEC, BOG, CON, DFO, EMT, EON, EZT, FAU, IFR, ISV, IWR, IWV, IWW, LTL, MAZ, MEI, MOR, MOZ, MTW, OFF, SFE, SFO, SSY, AEL, AFO, EUO, FER, HEU, LAU, MEL, MFI, MFS, MTT, MWW, NES, SFF, SFG, STF, STI, SZR, TER, TON or ERI. The zeolite preferably has one of the following framework structures: AFI, BEA, BOG, CON, EMT, EON, FAU, IWW, MAZ, MFI, MOR, MTW, OFF, SFE, SFO, SSY, AEL, EUO, FER, HEU, MEL, MFI, MTT, MWW, NES, STI, TON or ERI. The zeolite more preferably has one of the following framework structures: AFI, BEA, EMT, FAU, MFI, MOR, MTW, AEL, EUO, FER, HEU, MEL, MFI, MTT, MWW, NES, TON or ERI. These framework structures are described in “Atlas of Zeolite Framework Types” (Ch. Baerlocher, W.M. Meier, D.H. Olson, Elsevier, Sixth Revised Edition, 2007), the disclosure of which in this regard is incorporated into the description.

In one embodiment, the catalyst of the second catalyst layer comprises, as active component, tungstenated zirconium oxide or sulfated zirconium oxide, and has been promoted with a transition element or rare earth element.

The support of this catalyst can be generated by extrusion, tableting, spherization, pelletization, injection moulding or rapid prototyping methods.

In one embodiment, the second catalyst has an immobilized acid or ionic liquid on the support.

In one embodiment, the acidic or basic active component is incorporated into a permeable polymer matrix for production of a membrane. Thus, after application of the precious metal component to the porous support, use in a membrane reactor is possible.

Furthermore, the active component may be applied in the form of a washcoat to honeycombs, structured metal foils or column packing materials. The column packing materials may be placed in a column in a random or structured manner. Thus, after application of the precious metal component, use in a reactive distillation or in a microstructure reactor is possible.

The invention further relates to a process for catalytic hydroisomerisation of hydrocarbon mixtures in the presence of aromatics, olefins, organic sulfur compounds, organic nitrogen compounds, carbon monoxide, carbon dioxide, carbonyl sulfide or carbon disulfide or mixtures thereof, using the arrangement of the invention, wherein the process comprises the following steps:
providing a reactor for the hydroisomerisation;
arranging at least two catalyst layers, wherein the first catalyst layer is arranged upstream and the second catalyst layer is arranged downstream, and wherein the catalyst of the first catalyst layer is a supported precious metal catalyst for a hydrogenation of the reaction fluid and the catalyst of the second catalyst layer is a bifunctional supported precious metal catalyst, the support of which has acidic or basic properties, for the isomerisation of the reaction fluid after passing through the first catalyst layer,
charging the reactor with a hydrocarbon mixture;
converting the hydrocarbon mixture under hydroisomerisation conditions;
discharging the generated hydroisomerised hydrocarbon from the reactor.

The inlet temperature is the temperature that the hydrocarbon mixture has on entry into the reactor. This is typically in the range from 220 to 320° C., preferably in the range from 220 to 260° C., more preferably in the range from 230 to 250° C., most preferably in the range from 235 to 245° C.

The outlet temperature is the temperature that the product stream has on exit from the reactor. This is typically in the range from 240 to 340° C., preferably in the range from 240 to 300° C., more preferably in the range from 250 to 300° C., even more preferably in the range from 255 to 295° C., most preferably in the range from 265 to 295° C.

The reaction fluid introduced into the reactor comprises C4+ hydrocarbons, i.e. hydrocarbons having at least 4 carbon atoms in the structure. In one embodiment, the reaction fluid is a light gasoline fraction. A light gasoline fraction is understood by the person skilled in the art to mean a mixture of C4-C8 hydrocarbons, i.e. hydrocarbons having at least 4 carbon atoms to at most 8 carbon atoms. Light gasoline typically features an initial boiling point of at least 20° C. and a final boiling point of at most 95° C., measured to ASTM D86. In a further embodiment, the reaction fluid is a kerosene fraction. In a further embodiment, the reaction fluid is a mixture of hydrocarbons having an initial boiling point of 50° C. and an average boiling temperature of at most 200° C. In a further embodiment, the reaction fluid is a diesel fraction.

The hydrocarbon mixture entering the reactor may, as well as the hydrocarbons to be hydroisomerised, contain impurities and by-products.

For instance, the sulfur content is up to 10 000 ppm, preferably up to 5000 ppm, especially preferably up to 1000 ppm, more preferably from 50 to 1000 ppm. In one embodiment, the sulfur content is in the range from 100 to 10 000 ppm, preferably in the range from 500 to 5000 ppm, more preferably in the range from 500 to 1000 ppm.

The nitrogen content in the hydrocarbon mixture is typically in the range from 1 to 100 ppm, preferably in the range from 5 to 10 ppm.

The proportion of aromatics in the hydrocarbon mixture is typically up to 7%, especially up to 5%, and is preferably in the range from 1% to 5%.

In the process, hydrogenation of the impurities and by-products takes place in the first catalyst layer; hydroisomerisation of the hydrocarbons takes place in the second catalyst layer.

The product stream discharged from the reactor may, as well as the hydroisomerised hydrocarbons, also contain by-products and unconverted hydrocarbons.

In one embodiment, the process is a process for hydroisomerisation of aromatics to alkylated methylcyclopentanes.

In a further embodiment, the process of the invention results in a change in the boiling curve and density of the reaction fluid introduced into the reactor by cracking reactions or rearrangement reactions.

The process can be performed in a reactor housing or in separate reactor housings arranged in succession. The at least two catalyst layers lie. The catalyst layers may be present in the same reactor housing, or they are arranged separately from one another in reactor housings arranged in succession.

In one embodiment of the process, the at least two catalyst layers are present in separate columns or separately as column packing materials in a single distillation plant for the reactive distillation. The column packing materials may be placed in the distillation plant in a random or structured manner.

In a further embodiment, the at least two catalyst layers are present separately in a microstructure reactor or in separate microstructure reactors.

In a further embodiment, the at least two catalyst layers are in the form of a catalytically active membrane in a membrane reactor.

In a further embodiment of the process, layers of inert materials are additionally positioned above, between and/or beneath the catalyst layers. These may take the form of fixed reactor internals or of beds of inert material. These layers may serve to achieve better distribution of the components of the reaction fluid in the reactor, or to prevent catalyst material introduced into the reactor from falling out of it. In a preferred embodiment, the inert material is present beneath the second catalyst layer.

Suitable inert materials are preferably aluminium oxide, ceramics, fired silicon dioxide or fireclay.

In a further embodiment of the process, there is also one or more further catalyst layers beyond the catalyst layer arranged downstream or the layer of inert material optionally arranged beyond it.

For example, this further catalyst layer may comprise a catalyst for hydrodesulfurization in order to remove sulfur impurities present.

The present invention further provides for the use of the catalyst arrangement of the invention for catalytic hydroisomerisation of hydrocarbon mixtures in the presence of aromatics, olefins, organic sulfur compounds, organic nitrogen compounds, carbon monoxide, carbon dioxide, carbonyl sulfide or carbon disulfide or mixtures thereof.

The invention is described in detail hereinafter by multiple examples with reference to the appended drawings. The drawings show:

FIG. 1 a schematic diagram of an arrangement of the catalyst layers in a reactor

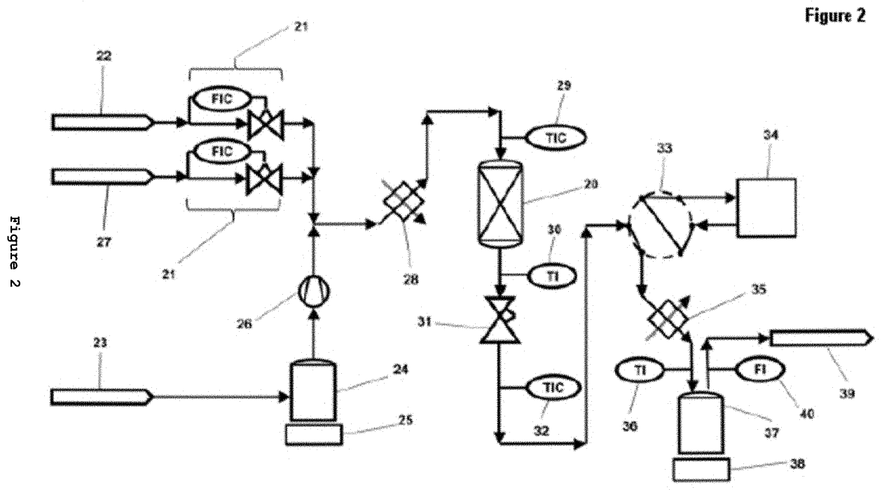

FIG. 2 a schematic diagram of a flow apparatus for performance of a process of the invention for hydroisomerisation of hydrocarbons

### EXAMPLES

The determinations of ignition loss in the context of the present invention were effected to DIN 51081 by determining the weight of about 1-2 g of a sample of the material to be analysed, then heating it to 900° C. under ambient atmosphere and storing it at this temperature for 3 h. Subsequently, the sample was cooled down under protective atmosphere and the remaining weight was measured. The difference in weight before and after thermal treatment corresponds to the ignition loss.

Experimental Apparatus

The comparative examples and inventive examples were performed using an experimental apparatus as described in FIG. 2. The setup was chosen for virtually adiabatic characteristics of the reactor. The dimensions of the reactor (20) were such that it could accommodate a total catalyst volume of at least 2500 cm³. It was also designed such that it could be operated at an operating pressure of 15 to 30 bar gauge.

For exact control of the volume flow rates, standard electronic mass flow regulators, called flow indication and controls FIC (21), were used. Nitrogen (22) served the purpose merely of purging of the plant in order that no explosive air-hydrogen or air-hydrocarbon mixtures could form. The feed oil (23) was initially charged in a cooled vessel (24) that rested on a balance (25) and was pumped by means of a pump (26) together with the hydrogen (27) into the crossflow microscale heat exchanger I (28). The crossflow microscale heat exchanger (28) was chosen such that it was possible to heat a hydrogen stream up to 400° C. in the above-specified pressure range of 1.5 kg/h (min. 5 kW). The pipelines to the reactor (20) were heated by means of temperature control by a temperature indicator and controller TIC (29) such that the desired reactor inlet temperature was maintained. At the reactor outlet was a thermocouple (30) for determining the reactor outlet temperature. The operating pressure was adjusted using a backpressure control valve (31). The reduced-pressure reaction fluid was guided in a pipe connection heated by means of temperature control by a temperature indicator controller TIC (32) to a sample loop (33) in order to analyse the composition of the reaction fluid with the aid of an online gas chromatograph (34). Alternatively, the sample loop connection permitted constant connection of a pipe connection to the crossflow microscale heat exchanger II (35). The reaction fluid was cooled to at least −10° C. by means of temperature controller (36) in order to collect an integral sample for further characterizations in the liquid sampling vessel (37), which likewise rested on a balance (38) to ascertain a mass balance. The escaping gas was supplied to an offgas conduit (39), determining the mass flow rate with a flow indicator FI (40).

The calculation of the yield Y, i.e. the product fraction based on molecules having a carbon number≥4, was found as the quotient of the mass m(C4+)_{liq} of the molecules having a carbon number≥4 that were collected in the vessel (37), and the mass m(C4+)_{gas} of the molecules having a carbon number≥4 in the offgas stream, which is determined by means of gas chromatography, divided by the mass m(C4+)inlet of the molecules having a carbon number≥4 that were initially charged in the vessel (24):

$$Y = \frac{m ( C 4 + ) liq + m ( C 4 + ) gas}{m ( C 4 + ) inlet}$$

The proportions by weight reported in tables 1 to 5 are each based on the total weight of the C4+ hydrocarbons present in the corresponding sample.

For comparative examples 1 and 2 and inventive examples 1 to 3, two light gasoline fractions were used: the olefin-free feed oil A and the olefin-containing feed oil B. The composition and some calculated properties are compiled in table 2.

TABLE 2
Composition and properties of the feed oils used (RON_{THEO}:
research octane number calculated from the composition)

| | Unit | Feed oil A | Feed oil B |
|---|---|---|---|
| n-Butane | % by weight | 5.63 | 5.63 |
| Isobutane | % by weight | 0.31 | 0.31 |
| n-Pentane (n-Pn) | % by weight | 35.63 | 35.23 |
| Isopentane (i-Pn) | % by weight | 4.46 | 4.46 |
| Neopentane | % by weight | 0.00 | 0.00 |
| Cyclopentane | % by weight | 3.63 | 3.63 |
| n-Hexane | % by weight | 17.45 | 17.45 |
| 2,2-Dimethylbutane | % by weight | 0.61 | 0.61 |
| 2,3-Dimethylbutane | % by weight | 1.76 | 1.76 |
| 2-Methylpentane | % by weight | 12.89 | 12.89 |
| 3-Methylpentane | % by weight | 7.81 | 7.81 |
| Cyclohexane | % by weight | 1.35 | 1.35 |
| Methylcyclopentane | % by weight | 6.26 | 6.26 |
| n-Heptane | % by weight | 0.10 | 0.10 |
| iso-Heptanes | % by weight | 0.10 | 0.10 |
| Benzene | % by weight | 1.99 | 1.99 |
| Toluene | % by weight | 0.00 | 0.00 |
| 1-Pentene | % by weight | 0.00 | 0.40 |
| Density at 15° C. | kg/dm³ | 0.657 | 0.657 |
| Average molecular mass | g/mol | 78.02 | 78.01 |
| RON_{THEO} | a.u. | 66.90 | 67.00 |
| iPn/(iPn + nPn) | % | 11.12 | 11.24 |

### Comparative Example 1

The reactor was charged with 1790 g of a commercially available zeolite catalyst, HYSOPAR®-5000 in extrudate form with an average diameter of 1.6 mm and a Pt content of 0.35% by weight from Clariant. The catalyst bed was positioned on an aluminium oxide bed consisting of tablets of dimensions 4.75×4.75 mm.

After the reactor had been filled, it was sealed pressure tight, and the plant was purged with a nitrogen stream of at least 500 dm³ (STP)/h versus ambient pressure for one hour. Subsequently, the nitrogen stream and the backpressure regulator were adjusted such that the same gas flow rate was attained at 30 bar gauge. After ten minutes, the gas supply was stopped in order to check the system for leaks. Subsequently, this procedure was repeated with hydrogen. For drying and activation of the catalyst, the reactor inlet temperature was first increased to 150° C. over a period of three hours under a hydrogen gas flow rate of 1000 dm³ (STP)/h versus ambient pressure. Subsequently, this temperature was maintained for a further three hours. This was followed by a constant increase in the reactor inlet temperature to 300° C. over a period of eight hours. This temperature was subsequently maintained for a further three hours.

Before the start of the catalytic experiment, the reactor inlet temperature was reduced to 200° C. at a constant cooling rate of 1 K/min and the hydrogen flow rate was adjusted to 905 dm³ (STP)/h versus 20 bar gauge.

At the start of the catalytic experiment, the olefin-free feed oil A was supplied at a mass flow rate of 2.628 kg/h and the temperature at the reactor inlet was increased from 200° C. to a first target temperature. After attainment of this temperature, these conditions were not changed over a period of three hours, and then the temperature at the reactor inlet was increased by a desired temperature. The number of possible gas chromatography analyses was determined by the necessary separation time. Typically, three injections were possible within three hours.

### Comparative Example 2

The reactor charge, procedure and experimental conditions corresponded to those of comparative example 1, except that the olefin-containing feed oil B was used.

### Example 1

The reactor was charged with 1432 g of a commercially available zeolite catalyst, HYSOPAR®-5000 in extrudate form with an average diameter of 1.6 mm and a Pt content of 0.35 Pt from Clamant. In addition, a further bed consisting of 250 kg of HYSOPAR®-1000 type catalyst in the form of a porous, weakly acidic aluminium oxide and with a Pt content of 0.30% by weight was introduced onto this catalyst bed. The bed of the HYSOPAR®-5000 catalyst was positioned on an aluminium oxide bed of tablets of dimensions 4.75×4.75 mm.

The procedure and experimental conditions corresponded to those of experimental example 1; the olefin-free feed oil A was likewise used.

### Example 2

The reactor was charged with 1432 g of a commercially available zeolite catalyst, HYSOPAR®-5000 in extrudate form with an average diameter of 1.6 mm and a Pt content of 0.35% by weight from Clamant. In addition, a further bed consisting of 250 kg of HYSOPAR®-1000 catalyst in the form of a porous, weakly acidic aluminium oxide and with a Pt content of 0.30% by weight from Clariant was introduced onto this catalyst bed. The bed of the HYSOPAR®-5000 catalyst was positioned on an aluminium oxide bed of tablets of dimensions 4.75×4.75 mm.

The procedure and experimental conditions corresponded to those of comparative example 1, except that the olefin-containing feed oil B was used.

### Example 3

The reactor was charged with 1432 g of a commercially available zeolite catalyst, HYSOPAR®-5000 in extrudate form with an average diameter of 1.6 mm and a Pt content of 0.25% by weight from Clariant. In addition, a further bed of 250 kg of HYSOPAR®-1000 catalyst in the form of a porous, weakly acidic aluminium oxide and with a Pt content of 0.30% by weight from Clariant was introduced onto this catalyst bed. The bed of the HYSOPAR®-5000 catalyst was positioned on an aluminium oxide bed of tablets of dimensions 4.75×4.75 mm.

The procedure and conditions corresponded to those of comparative example 1, except that the olefin-containing feed oil B was used.

Table 3 collates the results from the analysis of liquid products that were generated at different reactor inlet temperatures. The results show that, in the case of the inventive examples, higher yields were already achieved at lower inlet temperatures than in the comparative examples. Moreover, this result required a smaller amount of costly platinum overall.

TABLE 1
Summary of the reactor temperatures and the essential properties of the resultant product
streams from comparative examples 1 and 2 and inventive examples 1 to 3:

| | Comparative example | | Example | | |
|---|---|---|---|---|---|
| | 1 | 2 | 1 | 2 | 3 |
| Feed oil | A | B | A | B | B |
| Inlet temperature ° C. | 255 | 265 | 245 | 242 | 242 |
| Outlet temperature ° C. | 270 | 285 | 260 | 260 | 260 |
| Proportion of the following | | | | | |
| hydrocarbons [% by weight] | | | | | |
| n-Butane | 2.95 | 3.00 | 2.91 | 2.90 | 2.90 |
| Isobutane | 3.30 | 3.26 | 3.33 | 3.33 | 3.33 |
| n-Pentane | 14.40 | 15.44 | 14.37 | 14.36 | 14.36 |
| Isopentane | 26.39 | 25.33 | 26.44 | 26.45 | 26.45 |
| Neopentane | 0.00 | 0.00 | 0.00 | 0.00 | 0.00 |
| Cyclopentane | 2.67 | 2.66 | 2.67 | 2.67 | 2.67 |
| n-Hexane | 10.11 | 10.34 | 9.93 | 9.91 | 9.90 |
| 2,2-Dimethylbutane | 6.76 | 6.64 | 7.02 | 7.04 | 7.07 |
| 2,3-Dimethylbutane | 3.22 | 3.58 | 3.24 | 3.25 | 3.25 |
| 2-Methylpentane | 13.24 | 13.01 | 13.24 | 13.24 | 13.24 |
| 3-Methylpentane | 8.48 | 8.22 | 8.37 | 8.36 | 8.35 |
| Cyclohexane | 2.82 | 2.62 | 2.82 | 2.82 | 2.82 |
| Methylcyclopentane | 5.56 | 5.57 | 5.55 | 5.55 | 5.55 |
| n-Heptane | 0.06 | 0.06 | 0.06 | 0.06 | 0.06 |
| iso-Heptanes | 0.06 | 0.06 | 0.06 | 0.06 | 0.06 |
| Benzene | 0.00 | 0.20 | 0.00 | 0.00 | 0.00 |
| Toluene | 0.00 | 0.00 | 0.00 | 0.00 | 0.00 |
| Density [kg/dm³] | 0.6565 | 0.6566 | 0.6564 | 0.6564 | 0.6564 |
| RON [a.u.] | 78 | 78 | 78 | 79 | 79 |
| Yield [% by weight] | 95 | 92 | 96 | 97 | 97 |

### Example 4

The reactor was charged with 860 g of a commercially available zeolite catalyst, HYSOPAR®-7000 in extrudate form with an average diameter of 1.6 mm and a Pt content of 0.25% by weight from Clariant. In addition, a further bed of 900 g of HYSOPAR®-1000 catalyst in the form of a porous, weakly acidic aluminium oxide and with a Pt content of 0.30% by weight from Clariant was introduced onto this catalyst bed. The bed of the HYSOPAR®-5000 catalyst was positioned on an aluminium oxide bed of tablets of dimensions 4.75×4.75 mm.

The procedure corresponded to that of comparative example 1, except that the hydrogen flow rate was adjusted to 839 dm³ (STP)/h versus 30 bar gauge, and a benzene-containing feed oil C with the following composition and properties was used:

Feed oil C: 94% by weight of n-hexane and 6% by weight of benzene
RON_{THEO}=32
Density at 15° C.=0.6811 kg/dm³
Average molecular mass 98.875 g/mol

Table 4 collates the results from the analysis of liquid products that were generated in two experimental procedures A and B at different reactor inlet temperatures.

TABLE 4
Summary of the reactor temperatures and the essential properties
of the product streams obtained from example 4

| Parameter | Unit | A | B |
|---|---|---|---|
| Inlet temperature | ° C. | 220 | 240 |
| Outlet temperature | ° C. | 270 | 290 |
| Benzene | % by weight | 0 | 0 |
| Cyclohexane | % by weight | 2 | 2 |
| Methylcyclopentane | % by weight | 3 | 3 |
| n-Hexane | % by weight | 34 | 32 |
| i-Hexane | % by weight | 61 | 61 |
| C4-05 alkanes | % by weight | 0 | 2 |
| Yield | % by weight | 97 | 93 |
| RON | a.u. | 63 | 62 |
| Density | kg/dm³ | 0.6672 | 0.6674 |

It can be seen from the data from table 4 that the arrangement of the invention enables lowering of the inlet temperature with simultaneously improved yield and elevated RON.

### Example 5

The catalyst and procedure corresponded to those of example 4, except that a feed oil D having the following composition and properties was used:
Feed oil D: Kerosene fraction having a density of 0.7691 kg/dm³ at 15° C., 30 ppm by weight of sulfur and simulated boiling characteristics to ASTM D-2887 as in table 5.

TABLE 5
Boiling curve to ASTM D-2887 for the feed oil D used

| Boiling progression in % by weight | Temperature [° C.] |
|---|---|
| Start | 98.00 |
| 5 | 140.30 |
| 10 | 158.70 |
| 20 | 175.40 |
| 30 | 185.40 |
| 50 | 204.30 |
| 70 | 227.60 |
| 80 | 237.70 |
| 90 | 255.30 |
| 95 | 266.10 |
| End | 287.50 |

According to M. R. Riazi, Characterization and Properties of Petroleum Fractions, ASTM (2005) 1st edition, page 131, the “freeze point” is calculated from boiling progression and density to be FRP=−35° C.

The experimental conditions corresponded to those of example 4.

Table 6 collates the results from the analysis of liquid product streams that were generated in experimental procedures A, B and C at different reactor inlet temperatures.

TABLE 6
Summary of the reactor temperatures and the essential properties
of the product streams obtained from example 5

| | A | B | C |
|---|---|---|---|
| Inlet temperature [° C.] | 200 | 250 | 280 |
| Outlet temperature [° C.] | 240 | 290 | 320 |
| Boiling progression in % by weight | Temperature [° C.] | | |
| (to ASTM D-2887) | | | |
| Start | 95 | 48 | 22 |
| 5 | 133 | 125 | 48 |
| 10 | 152 | 144 | 89 |
| 90 | 255 | 254 | 246 |
| End | 287 | 288 | 289 |
| FRP [° C.] | -37 | -39 | -51 |
| Yield [% by weight] | 99.3 | 98.6 | 98.9 |
| Density [kg/dm³] | 0.7700 | 0.7694 | 0.7594 |

It can be seen from table 6 that the arrangement of the invention can achieve lowering of the FRP. It is also found that the yield of C4+ hydrocarbons can be increased when the process is performed at a lower inlet temperature.

1. Catalyst arrangement in a reactor for hydroisomerisation of hydrocarbons, wherein at least two catalyst layers are arranged in the reactor, wherein the first catalyst layer is arranged upstream and the second catalyst layer is arranged downstream, and wherein the catalyst of the first catalyst layer is a supported precious metal catalyst for a hydrogenation of the reaction fluid and the catalyst of the second catalyst layer is a bifunctional supported precious metal catalyst, the support of which has acidic or basic properties, for the isomerisation of the reaction fluid after passing through the first catalyst layer.

2. Catalyst arrangement according to claim 1, wherein the support of the catalyst of the first catalyst layer comprises an aluminium oxide, silicon oxide, a metal foam, ceramic or a thermally stable polymer.

3. Catalyst arrangement according to claim 1, wherein the catalyst of the second catalyst layer comprises, as active component, an amorphous aluminosilicate, zeolite, chlorinated aluminium oxide, tungstenated zirconium oxide or sulfonated zirconium oxide.

4. Catalyst arrangement according to claim 3, wherein the catalyst of the second catalyst layer comprises, as active component, tungstenated zirconium oxide or sulfated zirconium oxide, and has been promoted with a transition element or rare earth element.

5. Catalyst arrangement according to claim 1, wherein the downstream catalyst has an immobilized acid or ionic liquid on the support.

6. Catalyst arrangement according to claim 1, wherein the active component of the downstream catalyst has been embedded in a thermally stable organic, ceramic or metallic matrix by using a 3D printing method (rapid prototyping).

7. Catalyst arrangement according to claim 1, wherein the catalyst of the first catalyst layer and/or the catalyst of the second catalyst layer has a precious metal content within a range from 0.05% to 5.0% by weight, preferably from 0.1% to 4.0% by weight and more preferably from 0.1% to 3.0% by weight, based on the weight of the catalyst after ignition loss at 900° C.

8. Catalyst arrangement according to claim 1, wherein the catalyst layers are in the same reactor housing or separately from one another in reactor housings arranged in succession.

9. Use of the catalyst arrangement according to claim 1 for catalytic hydroisomerisation of hydrocarbon mixtures in the presence of aromatics, olefins, organic sulfur compounds, organic nitrogen compounds, carbon monoxide, carbon dioxide, carbonyl sulfide or carbon disulfide or mixtures thereof.

10. Process for catalytic hydroisomerisation of hydrocarbon mixtures in the presence of aromatics, olefins, organic sulfur compounds, organic nitrogen compounds, carbon monoxide, carbon dioxide, carbonyl sulfide or carbon disulfide or mixtures thereof, with a catalyst arrangement according to claim 1, wherein the process comprises the following steps:
providing a reactor for the hydroisomerisation;
arranging at least two catalyst layers, wherein the first catalyst layer is arranged upstream and the second catalyst layer is arranged downstream, and wherein the catalyst of the first catalyst layer is a supported precious metal catalyst for a hydrogenation of the reaction fluid and the catalyst of the second catalyst layer is a bifunctional supported precious metal catalyst, the support of which has acidic or basic properties, for the isomerisation of the reaction fluid after passing through the first catalyst layer,
charging the reactor with a hydrocarbon mixture;
converting the hydrocarbon mixture under hydroisomerisation conditions;
discharging the generated hydroisomerised hydrocarbon from the reactor.

11. Process according to claim 10 for the variation of the boiling curve and density of a hydrocarbon mixture by cracking reactions or rearrangement reactions.

12. Process according to claim 10 for hydroisomerisation of aromatics to alkylated methylcyclopentanes.

13. Process according to claim 10, wherein the at least two catalyst layers are present in separate columns or separately as column packing materials in a single distillation plant for the reactive distillation.

14. Process according to claim 10, wherein the two catalyst layers are present separately in a microstructure reactor or in separate microstructure reactors.

15. Process according to claim 10, wherein at least one of the two catalyst layers is in the form of a catalytically active membrane in a membrane reactor.

16. Process according to claim 10, wherein the inlet temperature is in the range from 220 to 320° C., preferably in the range from 220 to 260° C., more preferably in the range from 230 to 250° C., most preferably in the range from 235 to 245° C.

17. Process according to claim 10, wherein one or more further catalyst layers are arranged downstream of the catalyst layer arranged downstream.

18. Process according to claim 10, wherein the reaction fluid is a light gasoline fraction.
